(19) United States
(12) Patent Application Publication
(10) Pub. No.: US 2026/0176592 A1
(43) Pub. Date: Jun. 25, 2026

(54) VITRIFICATION AND REWARMING ASSEMBLIES AND METHODS

(71) Applicant: The University Of North Carolina At Charlotte, Charlotte, NC (US)

(72) Inventors: Charles Lee, Charlotte, NC (US); James Inman, Charlotte, NC (US); Madison Richards, Charlotte, NC (US); Alexander White, Charlotte, NC (US); Angel Koshy, Charlotte, NC (US); Guyrie Ferew, Charlotte, NC (US); Kateland Wilhite, Charlotte, NC (US); Micah Jenkins, Charlotte, NC (US); Christa Barsanti, Charlotte, NC (US); Kelvin Limanto, Charlotte, NC (US); Alexandria Traynham, Charlotte, NC (US)

(21) Appl. No.: 19/541,733

(22) Filed: Feb. 17, 2026

Related U.S. Application Data
(63) Continuation-in-part of application No. 19/174,511, filed on Apr. 9, 2025.
(60) Provisional application No. 63/635,796, filed on Apr. 18, 2024.

(51) Int. Cl. C12N 5/00

(52) U.S. Cl. CPC C12N 5/545; C12N 5/562

(57) ABSTRACT

A vitrification assembly for cryopreserving a biological sample, including a frame, a pair of vertically oriented, horizontally opposed cold plates, a cold plate actuator coupled to the pair of cold plates configured to control a distance between the pair of cold plates, a lift assembly configured to retain and raise and lower the biological sample between the pair of cold plates, a liquid nitrogen reservoir disposed below and around a bottom portion each of the pair of cold plates for cooling the pair of cold plates and the biological sample, and a control system configured to operate the cold plate actuator and monitor a temperature of the pair of cold plates and the biological sample. In some embodiments, the vitrification assembly further includes a heater thermally coupled to the pair of cold plates and operable for warming the pair of cold plates and the biological sample during a rewarming process.

### CROSS REFERENCE TO RELATED APPLICATIONS

The present disclosure is a continuation-in-part (CIP) of co-pending U.S. patent application Ser. No. 19/174,511, filed on Apr. 9, 2025, which claims the benefit of priority of co-pending U.S. Provisional Patent Application No. 63/635,796, filed on Apr. 18, 2024, the contents of both of which are incorporated in full by reference.

### TECHNICAL FIELD

The present invention disclosure relates generally to the cryopreservation field. More particularly, the present disclosure relates to vitrification and rewarming assemblies and methods for biological samples and the like, such as liver spheroid cells and the like.

### BACKGROUND

Vitrification is a method of cryopreservation in which biological materials are cooled so rapidly that ice crystals do not have time to form, thereby preventing damage. Conventional methods for vitrification suffer from limited throughput, inconsistent freezing rates, and difficulties in controlling critical parameters, such as plate temperature and spacing.

Liver spheroid cells, when used in research or therapeutic applications, require preservation methods that maintain cell viability and integrity. Thus, there exists a need for a vitrification assembly that is safe, reliable, precise, and capable of handling larger quantities with better automation and monitoring capabilities.

The present background is provided as illustrative environmental context only and should not be construed to be limiting in any manner. It will be readily apparent to those of ordinary skill in the art that the principles and concepts of the present disclosure may be implemented in other contexts equally, without limitation.

### SUMMARY

The present disclosure provides vitrification and rewarming assemblies and methods that overcome the limitations of conventional systems. These vitrification and rewarming assemblies may be separate assemblies or may be combined into the same package, representing separation operation modes or functionalities.

In some embodiments, the present disclosure provides a vitrification assembly for cryopreserving a biological sample, the vitrification assembly including a frame, a pair of vertically oriented, horizontally opposed cold plates disposed within the frame, a cold plate actuator coupled to the pair of cold plates and configured to control a distance between the pair of cold plates, a lift assembly configured to retain and raise and lower the biological sample between the pair of cold plates, a liquid nitrogen reservoir disposed below and around a bottom portion each of the pair of cold plates for cooling the pair of cold plates and the biological sample, and a control system configured to operate the cold plate actuator and monitor a temperature of the pair of cold plates and the biological sample via at least one thermocouple. In some embodiments, the cold plate actuator includes a stepper motor connected to a linkage system that moves the pair of cold plates in unison. In some embodiments, the bottom portion of each of the plurality of cold plates includes fins or other protruding structures that enhance thermal conductivity with liquid nitrogen disposed in the liquid nitrogen reservoir. In some embodiments, the at least one thermocouple includes a thermocouple associated with the biological sample and a thermocouple associated with at least one of the pair of cold plates. In some embodiments, the lift assembly includes a vertically movable carriage coupled a crossbar for holding the biological sample. In some embodiments, the lift assembly includes a pair of telescoping rods for vertically moving the biological sample. The vitrification assembly further includes an insulated housing disposed about the frame to reduce thermal gain. In some embodiments, the pair of cold plates are mounted to the cold plate actuator using a kinematic coupling system to accommodate thermal expansion differences between materials. In some embodiments, the control system is configured to automatically adjust spacing between the pair of cold plates during a vitrification cycle based on temperature data. In some embodiments, the vitrification assembly further includes a heater thermally coupled to the pair of cold plates and operable for warming the pair of cold plates and the biological sample during a rewarming process.

In some embodiments, the present disclosure provides a vitrification and rewarming assembly for cryopreserving and rewarming a biological sample, the vitrification and rewarming assembly including a frame, a pair of vertically oriented, horizontally opposed cold plates disposed within the frame, a cold plate actuator coupled to the pair of cold plates and configured to control a distance between the pair of cold plates, a lift assembly configured to retain and raise and lower the biological sample between the pair of cold plates, a liquid nitrogen reservoir disposed below and around a bottom portion each of the pair of cold plates for cooling the pair of cold plates and the biological sample, a heater thermally coupled to the pair of cold plates and operable for warming the pair of cold plates and the biological sample during a rewarming process, and a control system configured to operate the cold plate actuator and monitor a temperature of the pair of cold plates and the biological sample via at least one thermocouple.

In some embodiments, the present disclosure provides a rewarming assembly for rewarming a cryopreserved biological sample, the rewarming assembly including a frame, a pair of vertically oriented, horizontally opposed plates disposed within the frame, a plate actuator coupled to the pair of cold plates and configured to control a distance between the pair of plates, a lift assembly configured to retain and raise and lower the biological sample between the pair of plates, a heater thermally coupled to the pair of plates and operable for warming the pair of plates and the biological sample during a rewarming process, and a control system configured to operate the plate actuator and monitor a temperature of the pair of plates and the biological sample via at least one thermocouple. In some embodiments, the plate actuator includes a stepper motor connected to a linkage system that moves the pair of plates in unison. In some embodiments, the at least one thermocouple includes a thermocouple associated with the biological sample and a thermocouple associated with at least one of the pair of plates. In some embodiments, the lift assembly includes a vertically movable carriage coupled a crossbar for holding the biological sample. In some embodiments, the lift assembly includes a pair of telescoping rods for vertically moving the biological sample. The rewarming assembly further includes an insulated housing disposed about the frame to reduce thermal loss. In some embodiments, the pair of plates are mounted to the plate actuator using a kinematic coupling system to accommodate thermal expansion differences between materials. In some embodiments, the control system is configured to automatically adjust spacing between the pair of plates during a rewarming cycle based on temperature data. In some embodiments, the rewarming assembly further includes a liquid nitrogen reservoir disposed below and around a bottom portion each of the pair of plates for cooling the pair of plates and the biological sample.

It will be readily apparent to those of ordinary skill in the art that aspects and features of the described embodiments may be incorporated, omitted, or combined as desired in a given application, without limitation.

### BRIEF DESCRIPTION OF THE DRAWINGS

The present disclosure is illustrated and described with reference to the various drawings, in which like reference numbers are used to denote like assembly components/method steps, as appropriate.

FIG. 1 illustrates one embodiment of the vitrification/rewarming assembly of the present disclosure.

FIG. 2 illustrates the cooling rates of cryopreservant samples at various thermal lengths (° C./s).

FIG. 3 illustrates the warming rates of cryopreservant samples at various thermal lengths (° C./s).

FIG. 4 illustrates another embodiment of the vitrification/rewarming assembly of the present disclosure.

FIG. 5 illustrates an electrical schematic of one embodiment of the vitrification/rewarming assembly of the present disclosure.

It will be readily apparent to those of ordinary skill in the art that aspects and features of the illustrated embodiments may be incorporated, omitted, or combined as desired in a given application, without limitation.

### DETAILED DESCRIPTION

Again, the present disclosure provides vitrification and rewarming assemblies and methods that overcome the limitations of conventional systems. These vitrification and rewarming assemblies may be separate assemblies or may be combined into the same package, representing separation operation modes or functionalities.

Embodiments of the present disclosure are described in this detailed description. The present disclosure may be embodied in many different forms and should not be construed as being limited to the embodiments set forth; rather, these embodiments are provided so that the present disclosure satisfies applicable legal requirements.

The terminology used is for the purpose of describing particular embodiments only and is not intended to be limiting of the present disclosure. As used, the term “and/or” includes any and all combinations of one or more of the associated listed items. As used, the singular forms “a,” “an,” and “the” are intended to include the plural forms as well as the singular forms, unless the context clearly indicates otherwise. It will be further understood that the terms “comprises” and/or “comprising,” when used, specify the presence of the stated features, steps, operations, elements, and/or components, but do not preclude the presence or addition of one or more other features, steps, operations, elements, components, and/or groups.

Unless otherwise defined, all terms (including technical and scientific terms) used have the same meaning as commonly understood by one having ordinary skill in the art to which the present disclosure belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined.

In describing the present disclosure, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the present disclosure should be read with the understanding that such combinations are entirely within the scope of the present disclosure.

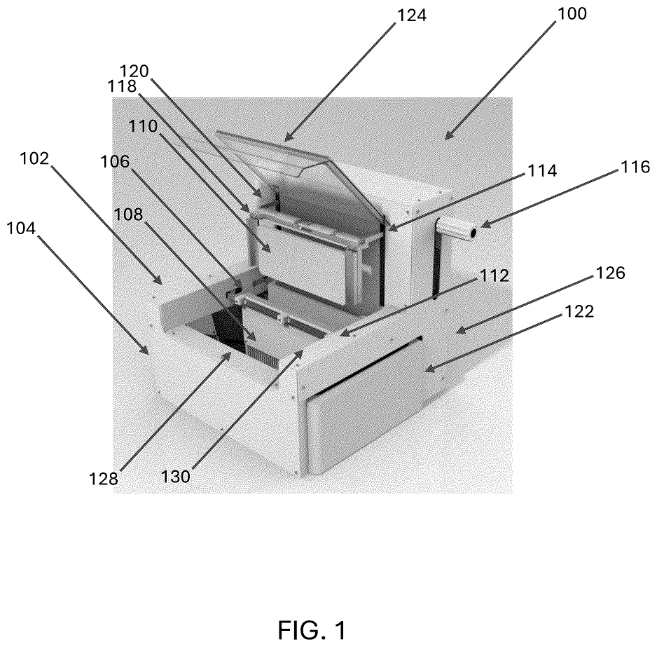

Referring to FIG. 1, in one embodiment, the vitrification assembly 100 includes a rigid frame 102 made of metal or the like, such as 1″×1″ aluminum extrusion members or the like, which serves as the structural backbone for all mounted components. This extrusion material is selected for its lightweight, low thermal conductivity, corrosion resistance, and ease of modular assembly. The design of the rigid frame 102 is such that it allows for independent subassemblies to be attached and removed for maintenance or upgrades. Each extrusion interface includes brackets and slot nuts that allow for rapid prototyping and final assembly.

The rigid frame 102 is thermally insulated with foam board and/or high-density polyethylene (HDPE) siding or the like 104 to prevent ambient heat from compromising the efficiency of the cooling system. HDPE and polycarbonate panels 104 are affixed to all sides of the rigid frame 104 using screws and thermal adhesive, for example. These materials are chosen due to their high insulation value and ability to withstand repeated exposure to cryogenic environments without embrittlement. Others suitable materials may be used equally.

The core operational function of the vitrification assembly 100 centers around two vertically oriented, horizontally opposed aluminum slam freeze plates (SFPs) or the like 106. These SFPs 106 are machined from 6061 T6 aluminum or the like for its thermal conductivity and cryogenic stability. Fins or other thermal enhancement structures 108 are machined onto/into the bottom surface or portion of each SFP 106 to increase the SFP surface area in contact with the liquid nitrogen bath in which the SFPs 106 are at least partially disposed. This enhanced SFP surface area improves heat transfer efficiency, allowing for faster and more uniform SFP cooling.

The face of each SFP 106 that contacts the vitrification sample 110 (e.g., IV bag) is precision machined to a flat tolerance, ensuring even compression across the vitrification sample surface. Uneven surfaces may cause air gaps, which act as insulators and diminish cooling efficiency. The flat face maintains intimate contact with the vitrification sample 110 during the vitrification process, allowing the solution inside to reach vitrification temperatures rapidly and uniformly.

The SFPs 106 may be mounted using a modified Maxwell kinematic coupling system that allows for thermal expansion differences between the aluminum plates and the HDPE substructure. Each SFP 106 is mounted at three points on stainless steel balls, which rest in corresponding grooves. This mounting system ensures consistent alignment and maintains the structural integrity of the vitrification assembly 100 at temperatures approaching −196° C.

The vitrification assembly 100 includes a cold plate actuator 112 including a stepper motor that drives a mechanical linkage system connected to two pairs of linear rods coupled to the SFPs 106. This cold plate actuator 112 serves to compress the SFPs 106 horizontally together into contact with the intervening vitrification sample 110 and expand the SFPs 106 apart out of contact with the intervening vitrification sample 110. The rods are case-hardened steel and ride in polymer linear bearings for consistent linear motion. The linkage system utilizes aluminum cranks and steel hubs mounted on the rear of the vitrification assembly 100, away from cryogenic exposure. The stepper motor itself includes a dual shaft, with one end driving the mechanical system and the other end attached to a manual adjustment knob or the like.

In some embodiments, the manual adjustment knob is geared with a 5:1 belt reduction system, allowing a user to manually control cold plate spacing with minimal torque. The stepper motor is electrically disengaged during manual mode to prevent back-driving or resistance. A control button toggles between automatic and manual modes, and an indicator LED shows the current setting.

In some embodiments, the cold plate actuator 112 allows for both coarse and fine adjustment of the spacing between the cold plates 106 to accommodate various vitrification sample or bag sizes and volumes. A key advantage of this design is the ability to programmatically release compression on the vitrification sample 110 near the end of the vitrification cycle. This slow decompression relieves internal stresses on the vitrified mass, which helps preserve the structural integrity of the preserved cells or the like.

The vitrification lift system 114 consists of a vertical elevator rail mounted adjacent to the cold plates 106. The elevator 114 is operated by a side-mounted manual handle 116 that raises or lowers the vitrification sample 100 between the cold plates 106. The elevator 114 locks automatically in the highest and lowest positions using a detent system and magnetic catches, ensuring secure placement before and after vitrification. Alternatively, the elevator 114 may be powered and have multiple detent positions.

In some embodiments, the IV bag 110 is suspended from stainless steel hooks 118 mounted on a detachable crossbar 120. This crossbar 120 connects magnetically to the bag lift carriage 114 and can be preloaded with the IV bag 110 prior to insertion. This design reduces handling time and minimizes the risk of premature warming or improper bag positioning during the transfer.

The liquid nitrogen tub 122 beneath the cold plates 106 is made of insulated stainless steel or the like and is detachable for cleaning or maintenance. It is surrounded on all sides by foam insulation and is connected to the main frame 102 using Velcro straps and thermal adhesive, for example. The tub 122 is filled until the nitrogen level reaches the bottom fins 108 of the SFPs 106, ensuring maximum thermal conductivity.

A foam insulated lid 124 is placed over the nitrogen bath during the cooling cycle to prevent rapid boil-off and to control condensation zones. By managing where condensation occurs, the vitrification assembly 100 avoids moisture buildup near electrical components, thus enhancing reliability and operational safety.

The electrical control system 126 is located in an insulated rear compartment isolated from the cryogenic chamber 128. This compartment contains a stepper motor driver, Arduino Mega microcontroller, breadboard-mounted thermocouple amplifiers, power supply, and USB output for data streaming. Components are mounted to a polycarbonate baseplate using nylon screws to minimize heat conduction.

Temperature sensing is achieved via two K-type thermocouples. One thermocouple is placed inside the vitrification sample 110 using a medical-grade needle and grommet. The second thermocouple is mounted on the face of the aluminum plate. Both are connected to Adafruit MAX31856 breakout boards, which transmit digitized temperature data to the Arduino controller.

The controller is programmed with custom firmware that streams real-time temperature data through a USB port to a connected PC. The PC uses Microsoft Excel with the Data Streamer add-in to graph and log temperatures. This system provides the operator with critical insights into the cooling performance and vitrification status without requiring additional instrumentation.

Control buttons may be mounted to the front panel: plate open, plate close, manual/auto toggle, and emergency stop. Each button is connected to a debounce circuit and a digital input pin on the controller. LED indicators adjacent to each button provide visual feedback for operational status.

In some embodiments, the vitrification assembly 100 supports programmable setpoints for automated opening and closing sequences. For example, at the beginning of the cycle, the cold plates 106 may be pre-set to a specific distance to accept a full 1000 mL IV bag 110. After cooling, the cold plates 106 may be incrementally separated to reduce mechanical stress. All movement sequences are encoded as part of the Arduino sketch and may be customized by modifying variables in the firmware.

In some embodiments, software safety interlocks are programmed into the control logic. These interlocks prevent motion commands if the controller detects unsafe conditions such as disconnected thermocouples, temperatures above −120° C., or lack of USB data streaming. These safety features ensure reliable performance and protect samples 110 from being lost or damaged due to user error.

In some embodiments, the vitrification assembly 100 includes manual override features to ensure usability in the event of power failure. The stepper motor can be disengaged, allowing the user to manually operate the cold plate actuator 112. All necessary tools for manual operation, such as the adjustment knob and thermocouple display, remain accessible on the exterior of the assembly 100.

All materials used are cryo-compatible and chosen for their strength and stability under thermal cycling. This includes stainless steel fasteners, nylon mounting hardware, and HDPE panels. All wiring is routed through a silicone-insulated loom to prevent embrittlement and electrical shorts under low temperatures.

The vitrification assembly 100 is designed for modular expansion. Additional thermocouples may be added, and the Arduino sketch includes configurable input channels to accommodate more sensors. This expandability is especially beneficial for advanced testing protocols involving multi-bag vitrification or varying bag geometries.

The entire vitrification assembly 100 is designed with manufacturability and scaling in mind. CAD drawings provided with the prototype allow for CNC machining or 3D printing of complex parts. Assembly is streamlined via subassemblies and modular wiring, and all major components can be disassembled using standard tools.

For rewarming the vitrification sample 110, heaters 130 may be thermally coupled to the cold plates 106 and used in the absence of the liquid nitrogen tub 122. It will be readily apparent to those of ordinary skill in the art that the vitrification and rewarming assemblies 100 may be separate, but similar, assemblies, utilizing separate, but similar, components. Alternatively, the vitrification and rewarming assemblies 100 may be a combined assembly, utilizing the heaters 130 coupled to the cold plates 106. In either case, this provides improved functionality and reliability for vitrification and rewarming for biological materials for cell therapy and the like. The rapid cooling of such biological materials from liquid to a glass-like state is provided without crystal formation. Cryopreservation solution protects the biological material during the vitrifying process. Samples are cooled above the critical cooling rate of 5° C./min and can be preserved indefinitely. Rewarming is above the critical warming rate of 50° C./min to avoid ice crystal formation that could destroy the biological material.

The SFPs 106 may be compressed onto the vitrification sample 110 at predetermined thermal thicknesses of 1.5 mm, 3 mm, or 5 mm, for example. Once the sample 110 is vitrified, the SFPs 106 may be opened and the sample 110 may be removed for remote storage.

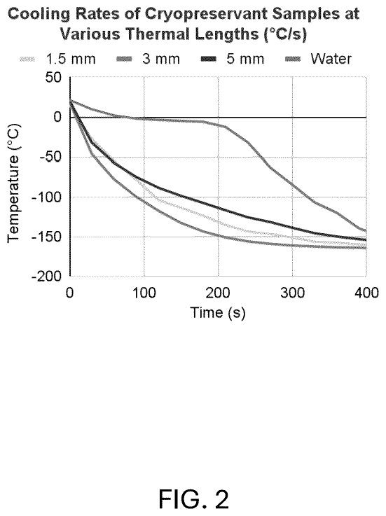

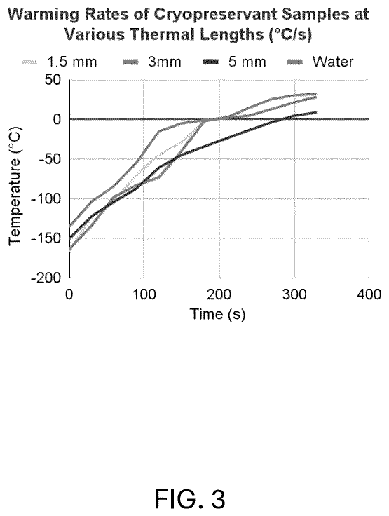

FIG. 2 illustrates the cooling rates of cryopreservant samples at various thermal lengths (° C./s). FIG. 3 illustrates the warming rates of cryopreservant samples at various thermal lengths (° C./s).

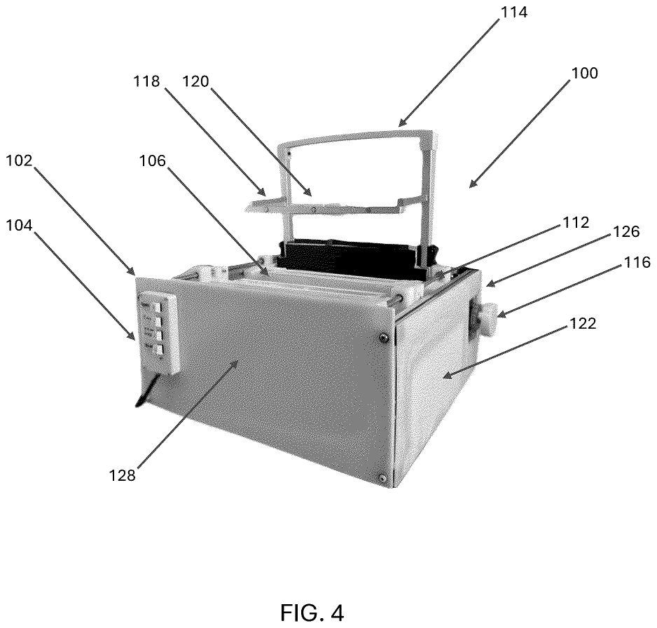

Referring to FIG. 4, in another embodiment, the vitrification assembly 100 again includes a rigid frame 102 made of metal or the like, such as 1″×1″ aluminum extrusion members or the like, which serves as the structural backbone for all mounted components. This extrusion material is selected for its lightweight, low thermal conductivity, corrosion resistance, and ease of modular assembly. The design of the rigid frame 102 is such that it allows for independent subassemblies to be attached and removed for maintenance or upgrades. Each extrusion interface includes brackets and slot nuts that allow for rapid prototyping and final assembly.

The rigid frame 102 is thermally insulated with foam board and/or HDPE siding or the like 104 to prevent ambient heat from compromising the efficiency of the cooling system. HDPE and polycarbonate panels 104 are affixed to all sides of the rigid frame 104 using screws and thermal adhesive, for example. These materials are chosen due to their high insulation value and ability to withstand repeated exposure to cryogenic environments without embrittlement. Others suitable materials may be used equally.

The core operational function of the vitrification assembly 100 centers around two vertically oriented, horizontally opposed aluminum SFPs or the like 106. These SFPs 106 are machined from 6061 T6 aluminum or the like for its thermal conductivity and cryogenic stability. Fins or other thermal enhancement structures 108 (FIG. 1) are machined onto/into the bottom surface or portion of each SFP 106 to increase the SFP surface area in contact with the liquid nitrogen bath in which the SFPs 106 are at least partially disposed. This enhanced SFP surface area improves heat transfer efficiency, allowing for faster and more uniform SFP cooling.

The face of each SFP 106 that contacts the vitrification sample 110 (FIG. 1) (e.g., IV bag) is precision machined to a flat tolerance, ensuring even compression across the vitrification sample surface. Uneven surfaces may cause air gaps, which act as insulators and diminish cooling efficiency. The flat face maintains intimate contact with the vitrification sample 110 during the vitrification process, allowing the solution inside to reach vitrification temperatures rapidly and uniformly.

The SFPs 106 may be mounted using a modified Maxwell kinematic coupling system that allows for thermal expansion differences between the aluminum plates and the HDPE substructure. Each SFP 106 is mounted at three points on stainless steel balls, which rest in corresponding grooves. This mounting system ensures consistent alignment and maintains the structural integrity of the vitrification assembly 100 at temperatures approaching −196° C.

The vitrification assembly 100 includes a cold plate actuator 112 including a stepper motor that drives a mechanical linkage system connected to two pairs of linear rods coupled to the SFPs 106. This cold plate actuator 112 serves to compress the SFPs 106 horizontally together into contact with the intervening vitrification sample 110 and expand the SFPs 106 apart out of contact with the intervening vitrification sample 110. The rods are case-hardened steel and ride in polymer linear bearings for consistent linear motion. The linkage system utilizes aluminum cranks and steel hubs mounted on the rear of the vitrification assembly 100, away from cryogenic exposure. The stepper motor itself includes a dual shaft, with one end driving the mechanical system and the other end attached to a manual adjustment knob or the like.

In some embodiments, the manual adjustment knob is geared with a 5:1 belt reduction system, allowing a user to manually control cold plate spacing with minimal torque. The stepper motor is electrically disengaged during manual mode to prevent back-driving or resistance. A control button toggles between automatic and manual modes, and an indicator LED shows the current setting.

In some embodiments, the cold plate actuator 112 allows for both coarse and fine adjustment of the spacing between the cold plates 106 to accommodate various vitrification sample or bag sizes and volumes. A key advantage of this design is the ability to programmatically release compression on the vitrification sample 110 near the end of the vitrification cycle. This slow decompression relieves internal stresses on the vitrified mass, which helps preserve the structural integrity of the preserved cells or the like.

The vitrification lift system 114 consists of a vertical elevator rail mounted adjacent to the cold plates 106. The elevator 114 is operated by a side-mounted manual handle 116 that raises or lowers the vitrification sample 100 between the cold plates 106. The elevator 114 locks automatically in the highest and lowest positions using a detent system and magnetic catches, ensuring secure placement before and after vitrification. Alternatively, the elevator 114 may be powered and have multiple detent positions.

In some embodiments, the IV bag 110 is suspended from stainless steel hooks 118 mounted on a detachable crossbar 120. This crossbar 120 connects magnetically to the bag lift carriage 114 and can be preloaded with the IV bag 110 prior to insertion. This design reduces handling time and minimizes the risk of premature warming or improper bag positioning during the transfer.

The liquid nitrogen tub 122 beneath the cold plates 106 is made of insulated stainless steel or the like and is detachable for cleaning or maintenance. It is surrounded on all sides by foam insulation and is connected to the main frame 102 using Velcro straps and thermal adhesive, for example. The tub 122 is filled until the nitrogen level reaches the bottom fins 108 of the SFPs 106, ensuring maximum thermal conductivity.

A foam insulated lid 124 (FIG. 1) is placed over the nitrogen bath during the cooling cycle to prevent rapid boil-off and to control condensation zones. By managing where condensation occurs, the vitrification assembly 100 avoids moisture buildup near electrical components, thus enhancing reliability and operational safety.

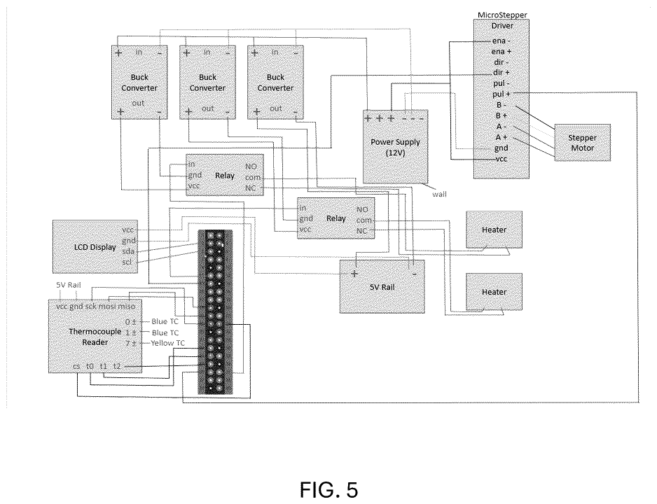

The electrical control system 126 is located in an insulated rear compartment isolated from the cryogenic chamber 128. This compartment contains a stepper motor driver, Arduino Mega microcontroller, breadboard-mounted thermocouple amplifiers, power supply, and USB output for data streaming. Components are mounted to a polycarbonate baseplate using nylon screws to minimize heat conduction. A schematic of the electrical system of the vitrification/rewarming assembly 100 is provided in FIG. 5.

Again, temperature sensing is achieved via two K-type thermocouples. One thermocouple is placed inside the vitrification sample 110 using a medical-grade needle and grommet. The second thermocouple is mounted on the face of the aluminum plate. Both are connected to Adafruit MAX31856 breakout boards, which transmit digitized temperature data to the Arduino controller.

The controller is programmed with custom firmware that streams real-time temperature data through a USB port to a connected PC. The PC uses Microsoft Excel with the Data Streamer add-in to graph and log temperatures. This system provides the operator with critical insights into the cooling performance and vitrification status without requiring additional instrumentation.

Control buttons may be mounted to the front panel: plate open, plate close, manual/auto toggle, and emergency stop. Each button is connected to a debounce circuit and a digital input pin on the controller. LED indicators adjacent to each button provide visual feedback for operational status.

For rewarming the vitrification sample 110, heaters 130 (FIG. 1) may be thermally coupled to the cold plates 106 and used in the absence of the liquid nitrogen tub 122. It will be readily apparent to those of ordinary skill in the art that the vitrification and rewarming assemblies 100 may be separate, but similar, assemblies, utilizing separate, but similar, components. Alternatively, the vitrification and rewarming assemblies 100 may be a combined assembly, utilizing the heaters 130 coupled to the cold plates 106. In either case, this provides improved functionality and reliability for vitrification and rewarming for biological materials for cell therapy and the like. The rapid cooling of such biological materials from liquid to a glass-like state is provided without crystal formation. Cryopreservation solution protects the biological material during the vitrifying process. Samples are cooled above the critical cooling rate of 5° C./min and can be preserved indefinitely. Rewarming is above the critical warming rate of 50° C./min to avoid ice crystal formation that could destroy the biological material.

The SFPs 106 may be compressed onto the vitrification sample 110 at predetermined thermal thicknesses of 1.5 mm, 3 mm, or 5 mm, for example. Once the sample 110 is vitrified, the SFPs 106 may be opened and the sample 110 may be removed for remote storage.

One of the desired functionalities of the vitrification/rewarming assembly 100 is the ability to test and operate at specific vitrification sample thicknesses or thermal lengths. The chosen solution is to set the thickness with the buttons that control the vitrification assembly 100 by setting the distance between the plates with a known value of 5 mm, and adjusting from there with 0.5 mm increments.

This ‘open all’ button works by running the motor that drives the plates to open until a limit switch is hit by one of the rails. This allows a consistent set point to be created. From there, it is a simple matter of determining how many steps the motor has to run to place the plates 5 mm apart from each other. This is the maximum thermal thickness that is desired to be tested, although other settings may be used equally. From there, buttons may be reassigned such that instead of an iterative open and close, there is now a button that takes the device from the open all position to the 5 mm position, and a button that moves the plates together in steps of 0.5 mm. This allows a range from entirely closed to 5 mm that can be tested in terms of thermal length.

The lift mechanism 114, especially for the rewarming assembly 100, utilizes telescoping rods to make the device more user-friendly, compact, stable, and lightweight. Telescoping rods are used because of their unique ability to compact and extend, their frequency in everyday life so non-engineers will intrinsically know how to operate them, their affordability, and their ability to be easily implemented into the current configuration. The telescoping rods may utilize an outer square rod of 1″ and an inner square rod of ⅞″ and a snap lock mechanism to hold them together.

The electronics may sit on a 3D-printed hanging shelf such that the electronics are easily accessible and removable. After some time attempting to print this shelf and get it installed securely and properly, the team decided that this was not the best course of action. Several issues were getting the shelf printed, and issues making the shelf sturdy enough to hold the weight of the electronics. The electronics may also sit in a box with sliding shelves. The box design may work better than the hanging shelf in some applications.

Preferably, the rewarming assembly 100 provides live temperature readings during the rewarming process.

Although the present disclosure is illustrated and described with reference to illustrative embodiments and examples, it will be readily apparent to those of ordinary skill in the art that other embodiments and examples may perform similar functions and/or achieve like results. All such equivalent embodiments and examples are within the spirit and scope of the present disclosure, are contemplated, and are intended to be covered by the following non-limiting claims for all purposes.

What is claimed is:

1. A vitrification assembly for cryopreserving a biological sample, the vitrification assembly comprising
a frame,
a pair of vertically oriented, horizontally opposed cold plates disposed within the frame,
a cold plate actuator coupled to the pair of cold plates and configured to control a distance between the pair of cold plates,
a lift assembly configured to retain and raise and lower the biological sample between the pair of cold plates,
a liquid nitrogen reservoir disposed below and around a bottom portion each of the pair of cold plates for cooling the pair of cold plates and the biological sample, and
a control system configured to operate the cold plate actuator and monitor a temperature of the pair of cold plates and the biological sample via at least one thermocouple.

2. The vitrification assembly of claim 1, wherein the cold plate actuator comprises a stepper motor connected to a linkage system that moves the pair of cold plates in unison.

3. The vitrification assembly of claim 1, wherein the bottom portion of each of the plurality of cold plates comprises fins or other protruding structures that enhance thermal conductivity with liquid nitrogen disposed in the liquid nitrogen reservoir.

4. The vitrification assembly of claim 1, wherein the at least one thermocouple comprises a thermocouple associated with the biological sample and a thermocouple associated with at least one of the pair of cold plates.

5. The vitrification assembly of claim 1, wherein the lift assembly comprises a vertically movable carriage coupled a crossbar for holding the biological sample.

6. The vitrification assembly of claim 1, wherein the lift assembly comprises a pair of telescoping rods for vertically moving the biological sample.

7. The vitrification assembly of claim 1, further comprising an insulated housing disposed about the frame to reduce thermal gain.

8. The vitrification assembly of claim 1, wherein the pair of cold plates are mounted to the cold plate actuator using a kinematic coupling system to accommodate thermal expansion differences between materials.

9. The vitrification assembly of claim 1, wherein the control system is configured to automatically adjust spacing between the pair of cold plates during a vitrification cycle based on temperature data.

10. The vitrification assembly of claim 1, further comprising a heater thermally coupled to the pair of cold plates and operable for warming the pair of cold plates and the biological sample during a rewarming process.

11. A vitrification and rewarming assembly for cryopreserving and rewarming a biological sample, the vitrification and rewarming assembly comprising
a frame,
a pair of vertically oriented, horizontally opposed cold plates disposed within the frame,
a cold plate actuator coupled to the pair of cold plates and configured to control a distance between the pair of cold plates,
a lift assembly configured to retain and raise and lower the biological sample between the pair of cold plates,
a liquid nitrogen reservoir disposed below and around a bottom portion each of the pair of cold plates for cooling the pair of cold plates and the biological sample,
a heater thermally coupled to the pair of cold plates and operable for warming the pair of cold plates and the biological sample during a rewarming process, and
a control system configured to operate the cold plate actuator and monitor a temperature of the pair of cold plates and the biological sample via at least one thermocouple.

12. A rewarming assembly for rewarming a cryopreserved biological sample, the rewarming assembly comprising
a frame,
a pair of vertically oriented, horizontally opposed plates disposed within the frame,
a plate actuator coupled to the pair of cold plates and configured to control a distance between the pair of plates,
a lift assembly configured to retain and raise and lower the biological sample between the pair of plates,
a heater thermally coupled to the pair of plates and operable for warming the pair of plates and the biological sample during a rewarming process, and
a control system configured to operate the plate actuator and monitor a temperature of the pair of plates and the biological sample via at least one thermocouple.

13. The rewarming assembly of claim 12, wherein the plate actuator comprises a stepper motor connected to a linkage system that moves the pair of plates in unison.

14. The rewarming assembly of claim 12, wherein the at least one thermocouple comprises a thermocouple associated with the biological sample and a thermocouple associated with at least one of the pair of plates.

15. The rewarming assembly of claim 12, wherein the lift assembly comprises a vertically movable carriage coupled a crossbar for holding the biological sample.

16. The rewarming assembly of claim 12, wherein the lift assembly comprises a pair of telescoping rods for vertically moving the biological sample.

17. The rewarming assembly of claim 12, further comprising an insulated housing disposed about the frame to reduce thermal loss.

18. The rewarming assembly of claim 12, wherein the pair of plates are mounted to the plate actuator using a kinematic coupling system to accommodate thermal expansion differences between materials.

19. The rewarming assembly of claim 12, wherein the control system is configured to automatically adjust spacing between the pair of plates during a rewarming cycle based on temperature data.

20. The rewarming assembly of claim 12, further comprising a liquid nitrogen reservoir disposed below and around a bottom portion each of the pair of plates for cooling the pair of plates and the biological sample.
